(19) United States
(12) Patent Application Publication
(10) Pub. No.: US 2024/0307423 A1
(43) Pub. Date: Sep. 19, 2024

(54) FRACTIONS OF, AND MOLECULES RELEASED IN, PROTEIN RICH PLASMA WHICH INHIBIT IMMUNE RESPONSES AND REDUCE INFLAMMATION, AND METHODS OF OBTAINING

(71) Applicant: NEW YORK SOCIETY FOR THE RELIEF OF THE RUPTURED AND CRIPPLED, MAINTAINING THE HOSPITAL FOR SPECIAL S, New York, NY (US)

(72) Inventors: Franck BARRAT, New York, NY (US); Marie Dominique AH KIOON, New York, NY (US)

(73) Assignee: NEW YORK SOCIETY FOR THE RELIEF OF THE RUPTURED AND CRIPPLED, MAINTAINING THE HOSPITAL FOR SPECIAL S, New York, NY (US)

(21) Appl. No.: 18/264,820

(22) PCT Filed: Feb. 10, 2022
(86) PCT No.: PCT/US2022/015961

§ 371 Date: Aug. 9, 2023

Related U.S. Application Data
(60) Provisional application No. 63/147,797, filed on Feb. 10, 2021.

(51) Int. Cl. A61K 31/7012; A61K 35/19; A61P 29/00

(52) U.S. Cl. CPC A61K 31/7012; A61K 35/19; A61P 29/00

(57) ABSTRACT

The current disclosure is related to decreasing or inhibiting inflammation and/or an immune response as well the prevention and treatment of diseases including autoimmune and musculoskeletal diseases, and injuries, using an active fraction of or a molecule isolated from protein rich plasma. The current disclosure also includes a method of obtaining an active fraction of protein rich plasma.

### CROSS REFERENCE TO RELATED APPLICATION

The present application claims priority to U.S. Patent Application Ser. No. 63/147,797 filed Feb. 10, 2021, which is hereby incorporated by reference in its entirety.

### BACKGROUND

Human platelets can impact the activation of other immune cells. This is important as these cells have a key function among other things in hemostasis and inflammation.

Protein rich plasma (PRP) is a concentrated of platelet-rich plasma protein derived from whole blood with is centrifuged to remove the red blood cells. PRP is widely used for a variety of disorders and conditions, including post-surgical healing, osteoarthritis, musculoskeletal disorders and cosmetic procedures, but with varying results.

It has been described that plasmacytoid dendritic cells (pDCs) are the main type I interferon (IFN) producers in human blood and they play a key role in innate anti-viral immunity. In addition, pDCs have been associated with autoimmunity in various contexts with a clear link with lupus (Barrat and Su 2019). pDCs and type I IFN also play a role in a number of cutaneous autoimmune diseases such as dermatomyositis, lichen sclerosis, cutaneous GVHD and cutaneous lupus (Wenzel and Tuting 2008).

N-Acetylneuraminic acid (otherwise known as Neu5AC or NANA) is a nine-carbon, sialic acid monosaccharide commonly found in glycoproteins on cell membranes and in glycolipids such as gangliosides in mammalian cells.

As shown herein, both PRP and N-Acetylneuraminic acid have an impact on immune cells and have anti-inflammatory properties.

### SUMMARY

The current disclosure is based upon several discoveries regarding platelets, platelet releasate and products released from platelets and their impact on the activation of immune cells.

Using in vitro systems with plasmacytoid dendritic cells (pDCs) as cells of interest, a series of surprising observations have been made. The platelet releasate (platelet-conditioned media or PCM which is a concentrated version of PRP) has a drastic effect on all the cell types tested with anti-inflammatory responses.

As shown herein PCM exerts an anti-inflammatory effect on TLR9- and TLR9+CXCL4-induced human plasmacytoid dendritic cell (pDC) activation. This PCM inhibits or decreases the production of interferon-α by the plasmacytoid dendritic cells. This effect is found in a fraction of PCM obtained using a method disclosed herein. The fraction is an acidic salt of the water phase of the PCM. As further shown herein, N-Acetylneuraminic acid or Neu5ac exerts this anti-inflammatory effect.

Thus, one embodiment of the current disclosure is a method of inhibiting or decreasing an immune response in a subject in need thereof comprising administering to the subject a therapeutically effective amount of N-Acetylneuraminic acid.

A further embodiment is a method for inhibiting or decreasing inflammation in a subject in need thereof comprising administering a therapeutically effective amount of N-Acetylneuraminic acid.

In the foregoing embodiments, the N-Acetylneuraminic acid inhibits or decreases the expression or production of interferon-α by plasmacytoid dendritic cells in the subject.

A further embodiment is a method for inhibiting or decreasing the production of interferon-α in plasmacytoid dendritic cells in a subject in need thereof comprising administering a therapeutically effective amount of N-Acetylneuraminic acid.

All of the foregoing methods would be beneficial to a subject suffering from, suspected of suffering from or at risk for an autoimmune disease.

A further embodiment of the present disclosure is a method of preventing and/or treating an autoimmune disease in a subject in need thereof comprising administering a therapeutically effective amount of N-Acetylneuraminic acid, wherein the N-Acetylneuraminic acid inhibits or decreases the expression or production of interferon-α by plasmacytoid dendritic cells in the subject.

In some embodiments, the autoimmune disease includes but is not limited to systemic lupus erythematosus, rheumatoid arthritis, type 1 diabetes, multiple sclerosis, myasthenia gravis, Graves disease, pernicious anemia, scleroderma, psoriasis, inflammatory bowel diseases, Hashimoto's disease, Addison's disease, cutaneous autoimmune disease, systemic sclerosis, and Sjögren's syndrome.

In these embodiments, the therapeutically effective amount of the N-Acetylneuraminic acid can be administered to the subject, for example, via injection, or the plasmacytoid dendritic cells can be treated, contacted or incubated with the N-Acetylneuraminic acid per the methods disclosed herein ex vivo, and transplanted into the subject.

The current disclosure also includes kits and pharmaceutical compositions comprising N-Acetylneuraminic acid alone or combined for use in the methods disclosed herein and comprising any of the cells treated, contacted or incubated as set forth by the disclosed methods for the treatment and/or prevention of diseases.

The current disclosure also includes a method of fractionating PCM to obtain an active fraction of the PCM/PRP, i.e., a fraction that inhibits or decreases inflammation and/or an immune response.

Thus, a further embodiment is a method of fractionating PCM to obtain an active fraction of the PCM comprising the steps of:
A. fractionating the PCM to obtain a water phase;
B. extracting the water phase using DMF under acidic condition, to obtain a solid acidic fraction; and
C. resolubilizing the solid acidic fraction in acetonitrile/methanol/H₂O (40/40/20) and collecting the soluble fraction to obtain the active liquid fraction.

Steps A and B of the foregoing method can be labor intensive and time consuming.

Thus, a streamlined and efficient method was developed to obtain the active fraction.

This method comprises the steps:
A. adding a mixture of DMF and 4M HCl in Dioxane at 4:1 by volume to freeze-dried platelet supernatant pellets and removing a soluble fraction to obtain a solid acidic fraction; and
B. resolubilizing the solid acidic fraction in acetonitrile/methanol/H₂O (40/40/20) and collecting the soluble fraction to obtain the active liquid fraction.

The disclosure also includes methods of using this active fraction of PCM or a salt in the active fraction of the PCM as a therapeutic or preventative agent.

A further embodiment is a method of inhibiting or decreasing an immune response in a subject in need thereof comprising administering to the subject a therapeutically effective amount of the active fraction of the PCM or a salt in the active fraction of the PCM.

A further embodiment is a method for inhibiting or decreasing inflammation in a subject in need thereof comprising administering a therapeutically effective amount of the active fraction of the PCM or a salt in the active fraction of the PCM.

In the foregoing embodiments, the active fraction of the PCM or the salt in the active fraction of the PCM inhibits or decreases the expression or production of interferon-α by plasmacytoid dendritic cells in the subject.

A further embodiment is a method for inhibiting or decreasing the production of interferon-α in plasmacytoid dendritic cells in a subject in need thereof comprising administering a therapeutically effective amount of the active fraction of the PCM or a salt in the active fraction of the PCM.

All of the foregoing methods would be beneficial to a subject suffering from, suspected of suffering from or at risk for an autoimmune disease.

A further embodiment of the present disclosure is a method of preventing and/or treating an autoimmune disease in a subject in need thereof comprising administering a therapeutically effective amount of the active fraction of the PCM or the salt in the active fraction of the PCM, wherein the active fraction of the PCM or the salt in the active fraction of the PCM inhibits or decreases the expression or production of interferon-α by plasmacytoid dendritic cells in the subject.

In some embodiments, the autoimmune disease includes but is not limited to systemic lupus erythematosus, rheumatoid arthritis, type 1 diabetes, multiple sclerosis, myasthenia gravis, Graves disease, pernicious anemia, scleroderma, psoriasis, inflammatory bowel diseases, Hashimoto's disease, Addison's disease, cutaneous autoimmune disease, systemic sclerosis, and Sjögren's syndrome.

Additionally, PRP is used for a variety of indications including enhancing the healing process especially with regard to the healing of muscles, tendons and ligaments. PRP injections are also used to treat orthopedic and musculoskeletal injuries and conditions such as soft tissue injuries, including but not limited to rotator cuff tears and Achilles tendon ruptures and other tendon injuries. PRP injections are also used to treat tendonitis and muscle strains and tears. PRP injections are also used for the treatment of osteoarthritis. PRP injections are also used for cosmetic procedures and for enhancing hair growth. As shown herein, N-Acetylneuraminic acid is found in the active fraction of the PCM and has the same anti-inflammatory effects of PCM/PRP. Thus, further embodiments of the current disclosure would include the use of N-Acetylneuraminic acid for any use currently PRP is currently indicated.

One embodiment is a method of treating musculoskeletal injuries and conditions in a subject in need thereof comprising administering a therapeutically effective amount of N-Acetylneuraminic acid.

Injuries would include but are not limited to soft tissue injuries including injuries to muscles, tendons and ligaments. Conditions would include but are not limited to osteoarthritis and tendonitis.

Also as shown herein, a particular active fraction of PCM and a salt in the active fraction of the PCM also have the same anti-inflammatory effects of PCM/PRP. Thus, further embodiments of the current disclosure would include the use of these for any use currently PRP is currently indicated.

One embodiment is a method of treating musculoskeletal injuries and conditions in a subject in need thereof comprising administering a therapeutically effective amount of the active fraction of PCM or a salt in the active fraction of the PCM.

Injuries would include but are not limited to soft tissue injuries including injuries to muscles, tendons and ligaments. Conditions would include but are not limited to osteoarthritis and tendonitis.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

Abbreviations that are used throughout the figures include: Med-medium alone used as control; HD-healthy donor; pDC-plasmacytoid dendritic cells; PMBC-peripheral mononuclear blood cells; PCM-platelet-conditioned media; TLR9-toll-like receptor 9; IFN-α-interferon-α.

FIG. 1 shows that PCM exerts an anti-inflammatory effect on TLR9+CXCL4-induced human plasmacytoid DC activation. FIG. 1A is a graph showing IFN-α secretion levels quantified in the supernatants by ELISA of purified pDCs (n=73) cultured in media alone (control), with CpG-B (TLR9 ligand), with CpG-B+CXCL4 or with the CpG-B+CXCL4 and PCM prepared from 15 different HDs for 24 hours. Values are represented as a mean±SEM and each dot represent an individual human HD. FIG. 1B shows the gene expression levels of IFN-α quantified by qPCR related to the housekeeping gene ubiquitin in purified pDCs (n=6) cultured in media alone (control), with CpG-B (TLR9 ligand), with CpG-B+CXCL4 or with the CpG-B+CXCL4 and PCM for 6 hours. Values are represented as a mean±SEM and each dot represent an individual human HD. Statistical significance was evaluated using a Mann-Whitney U-test and *p<0.05; **p<0.01; ***p<0.001.

FIG. 2 shows that PCM exerts an anti-inflammatory effect TLR9-induced human plasmacytoid DC activation. FIG. 2A is a graph showing IFN-α secretion levels quantified in the supernatants by ELISA of purified pDCs cultured in media alone (control), with CpG1018 (TLR9 ligand) 0.25 μM, or with the CpG1018 and PCM for 24 hours. Values are represented as a mean±SEM and each dot represent an individual human HD. FIG. 2B is a graph showing IFN-α secretion levels quantified in the supernatants by ELISA of purified pDCs cultured in media alone (control), with CpG1018 (TLR9 ligand) 1 μM, or with the CpG1018 and PCM for 24 hours. Values are represented as a mean±SEM and each dot represent an individual human HD. Statistical significance was evaluated using a Mann-Whitney U-test and *p<0.05; **p<0.01; ***p<0.001.

FIG. 3 shows that the molecule responsible for the anti-inflammatory effect in PCM on TLR9+CXCL4-activated human plasmacytoid DC is not a protein, nucleic acid or lipid. FIG. 3A is a graph showing IFN-α levels quantified in the supernatants by ELISA of purified pDCs cultured in media alone (control), with CpG-B (TLR9 ligand), with the CpG-B+CXCL4, with the CpG-B+CXCL4 and PCM, with the CpG-B+CXCL4 and hd PCM, and with the CpG-B+CXCL4 and PCM-PK. Values are represented as a mean±SEM and each dot represent an individual human HD. FIG. 3B is a graph showing IFN-α levels quantified in the supernatants by ELISA of purified pDCs cultured in media alone (control), with the CpG-B (TLR9 ligand), with the CpG-B+CXCL4, with the CpG-B+CXCL4 and PCM, with the CpG-B+CXCL4 and PCM first treated with RNase, and with the CpG-B+CXCL4 and PCM first treated with DNase. Values are represented as a mean±SEM and each dot represent an individual human HD. FIG. 3C is a graph showing IFN-α levels quantified in the supernatants by ELISA of purified pDCs cultured in media alone (control), with the CpG-B (TLR9 ligand), with the CpG-B+CXCL4, with the CpG-B+CXCL4 and PCM, and with the CpG-B+CXCL4 and PCM in which the lipids were removed. FIG. 3D is a graph showing IFN-α levels quantified in the supernatants by ELISA of purified pDCs cultured in media alone (control), with the CpG-B (TLR9 ligand), with the CpG-B+CXCL4, with the CpG-B+CXCL4 and PCM, with the CpG-B+CXCL4 and PCM with no proteins, nucleic acids or lipids. Values are represented as a mean±SEM and each dot represent an individual human HD. Statistical significance was evaluated using a Mann-Whitney U-test and *p<0.05; **p<0.01; ***p<0.001.

FIG. 4 is a graph showing IFN-α levels quantified in the supernatants by ELISA of purified pDCs cultured in media alone (control), with the CpG-B (TLR9 ligand), with the CpG-B+CXCL4, with the CpG-B+CXCL4 and PCM, and with the CpG-B+CXCL4 and PCM that was first separated into two different fractions: one of less than and one of more than 5 KDa using size exclusion filters. Values are represented as a mean±SEM and each dot represent an individual human HD. Statistical significance was evaluated using a Mann-Whitney U-test and *p<0.05; **p<0.01; ***p<0.001.

FIG. 5 shows the first fractionations of the platelet supernatant. FIG. 5A is a schematic of the first fractionations of the platelet supernatant. FIG. 5B is a graph of IFN-α production relative to CpG-CXCL4 in the various fractions showing that the water phase fraction designated F1 had inhibitory activity.

FIG. 6 is a schematic of the fractionation of the water phase of the PCM into six fractions.

FIG. 7 shows that solid acidic fraction designated F1.3 is the active fraction. FIG. 7A is a graph of IFN-α production relative to CpG-CXCL4 in the various fractions F1-1, F1-2, F1-3 and F1-4. FIG. 7B is a graph of IFN-α production relative to CpG-CXCL4 in the various fractions F1-5, F1-6 as well as the total fractions.

FIG. 8 is a schematic of the desalting and testing of the solid acidic phase.

FIG. 9 shows that the active component of the solid acidic fraction is most likely a salt and show that the fraction designated F1.3b is the active fraction. FIG. 9A is a graph of IFN-α production relative to CpG-CXCL4 in the various fractions F1.3, F1.3a, F1.3b, F1.3.1a, F1.3.1b, F1.3.2a, and F1.3.2b. FIG. 9B is a graph of IFN-α production relative to CpG-CXCL4 in the various dilutions of fractions F1.3a and F1.3b.

FIG. 10 shows that N-Acetylneuraminic acid (Neu5ac) has the inhibitory activity on the activated pDCs. FIG. 10A is a graph of IFN-α production relative to CpG-CXCL4 of various molecules including Neu5ac, 2-methylcitric (2MC) and α-Methyl-phenylalanine (CH3Ala). FIG. 10B is a graph of IFN-α production relative to CpG-CXCL4 of various molecules including Neu5ac, aspartyl glutamate (NAAG) and oxoglutaric acid (alpha-ketoglutaric acid) (αKG). FIG. 10C is a is a graph of IFN-α production relative to the TLR9 agonist CpG C274 of various dilutions of N-Acetylneuraminic acid.

FIG. 11 is schematic of a streamlined method to obtain the active fraction of the PCM.

### DETAILED DESCRIPTION

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “subject” as used in this application means an animal with an immune system such as avians and mammals. Thus, the invention can be used in veterinary medicine, e.g., to treat companion animals, farm animals, laboratory animals in zoological parks, and animals in the wild. The invention is particularly desirable for human medical applications.

The term “patient” as used in this application means a human subject. In some embodiments, the “patient” is one suffering with an autoimmune disease or suspected of suffering from or at risk for an autoimmune disease. In some embodiments, the “patient” is one suffering with a musculoskeletal disease, condition or injury or suspected of suffering from or at risk for a musculoskeletal disease, condition or injury. In some embodiments, the “patient” is one who would benefit from the inhibition or reduction of an immune response and/or inflammation.

The terms “treat”. “treatment”, and the like refer to a means to slow down, relieve, ameliorate or alleviate at least one of the symptoms of the disease or injury or defect, or reverse the disease or injury or defect after its onset.

The terms “prevent”, “prevention”, and the like refer to acting prior to overt disease onset, to prevent the disease from developing or minimize the extent of the disease or slow its course of development.

The phrase “therapeutically effective amount” is used herein to mean an amount sufficient to cause an improvement in a clinically significant condition in the subject, or delays or minimizes or mitigates one or more symptoms associated with the disease or injury or defect, or results in a desired beneficial change of physiology in the subject.

In some embodiments, the phrase “in need thereof” indicates a subject has, or is suspected of having, or has risk factors for a disease. In some embodiments, the disease is an autoimmune disease. In some embodiments, the disease is a musculoskeletal disease or condition In some embodiments, the phrase indicates the subject has an injury. In some embodiments, the phrase indicates a subject who would benefit from a reduction in inflammation and/or the inhibition or decrease in an immune response.

The term “agent” as used herein means a substance that produces or is capable of producing an effect and would include, but is not limited to, chemicals, pharmaceuticals, biologics, small organic molecules, antibodies, nucleic acids, peptides, and proteins.

The term “active” as used herein means having an effect. In some embodiments, this effect is to inhibit or reduce inflammation. In some embodiments, this effect is to inhibit or reduce an immune response.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

### Abbreviations

PRP—protein rich plasma
PCM—platelet conditioned media
pDC—plasmacytoid dendritic cells
IFN—interferon
kDa—kilodalton
Neu5ac—N-Acetylneuraminic acid
CpG—CpG-containing oligonucleotides
   An Active Fraction of PRP/PCM and N-Acetylneuraminic Acid have an Anti-Inflammatory Effect and Inhibit Immune Responses

To create platelet-rich plasma, clinicians take a blood sample from the patient and centrifuge it to separate out the other components of the blood from the platelets and concentrate them within the plasma. While injections of PRP are used for many indications, the mechanism behind them is not fully understood. It is believed that the increased concentration of growth factors in platelet-rich plasma may stimulate or speed up the healing process, shortening healing time for injuries, decreasing pain, and even encouraging hair growth.

As the results of using PRP are mixed, it would be of value to know not only which fraction of the PR/PCM is exerting the beneficial effects, but also the molecule.

As shown herein, platelet releasate including PRP and PCM, and more specifically a metabolite or molecule found in platelet releasate inhibits the inflammatory response of plasmacytoid dendritic cells (pDCs), decreasing or inhibiting the expression and/or secretion of IFN-α. Additionally, as shown herein, the acidic fraction of the water phase of the PCM is the active component of the PCM and it most likely a salt that has the activity.

PCM was fractionated and it was found that the water phase was the active fraction of the PCM. The water phase was further fractionated and the solid acidic fraction was found to be the active fraction. From there it was shown that the active molecule(s) was within a liquid fraction of the solid acidic fraction and was a salt.

Thus, one embodiment of the current disclosure the use of the active fraction of the PCM or a salt in the active fraction of the PCM to inhibit pDC cell activation.

A further embodiment of the current disclosure is the use of the active fraction of the PCM or a salt in the active fraction of the PCM to decrease or inhibit an immune response.

A further embodiment of the current disclosure the use of the active fraction of the PCM or a salt in the active fraction of the PCM to decrease or inhibit inflammation.

A further embodiment of the current disclosure the use of the active fraction of the PCM or a salt in the active fraction of the PCM to decrease or inhibit the production, expression and/or secretion of IFN-α from pDCs.

Yet a further embodiment of the current invention is a method of treating and/or preventing an autoimmune disease in a subject in need thereof by administering a therapeutically effective amount of the active fraction of the PCM or a salt in the active fraction of the PCM, wherein the administration of the active fraction of the PCM or the salt in the active fraction of the PCM decreases or inhibits the activation of pDCs to produce a lesser amount or level of IFN-α.

The further screening of several small molecules found in the salt fraction of the solid acidic fraction for anti-inflammatory/anti-immune response activity showed that N-Acetylneuraminic acid, otherwise known as Neu5AC, had this activity.

N-Acetylneuraminic acid (otherwise known as Neu5AC or NANA; IUPAC name: 5-(acetylamino)-3,5-dideoxy-D-glycero-α-D-galacto-non-2-ulopyranosonic acid) is a nine-carbon, sialic acid monosaccharide commonly found in glycoproteins on cell membranes and in glycolipids such as gangliosides in mammalian cells. Studies suggest that N-Acetylneuraminic acid is useful biologically in neurotransmission, leukocyte extravasation, viral or bacterial infections and carbohydrate-protein recognition.

As shown herein, N-Acetylneuraminic acid or Neu5ac inhibits the inflammatory and immune response of plasmacytoid dendritic cells (pDCs), decreasing or inhibiting their production, expression and/or secretion of IFN-α.

Thus, one embodiment of the current disclosure the use of Neu5ac to inhibit pDC cell activation.

A further embodiment of the current disclosure is the use of Neu5ac to decrease or inhibit an immune response.

A further embodiment of the current disclosure the use of Neu5ac to decrease or inhibit inflammation.

A further embodiment of the current disclosure the use of Neu5acC to decrease or inhibit the production, expression and/or secretion of IFN-α from pDCs.

pDC is the key cell type mediating TLR-induced inflammation in autoimmune disease patients as well. In lupus, it has been shown that pDCs produce large amounts of type I IFN due to TLR7 and TLR9 recognition of endogenous RNA and DNA in the form of immune complexes (Barrat et al. 2005). It has also been shown that pDC activation prevents optimal response to corticosteroid treatment by lupus patients (Guiducci et al. 2010), and two recent studies identified pDCs as the key cell type promoting lupus in mouse models of the disease (Sisirak et al. 2014; Rowland et al. 2014). The importance of pDCs has also been observed in a series of related cutaneous autoimmune diseases such as dermatomyositis, lichen sclerosis, cutaneous Graft-versus-host disease (GVHD) or cutaneous lupus that share a common pathological inflammatory feature described as “interface dermatitis” (Wenzel and Tuting 2008). In these patients, pDCs massively infiltrate the skin and produce IFN-α which plays a major role in the development of cutaneous lesions.

Thus, a further embodiment of the current invention is a method of treating and/or preventing an autoimmune disease in a subject in need thereof by administering a therapeutically effective amount of Neu5ac, wherein the administration of the Neu5ac decreases or inhibits the activation of pDCs to produce a lesser amount or level of IFN-α.

Autoimmune diseases that can be treated and/or prevented by the current methods of the invention include but are not limited to include but are not limited to systemic lupus erythematosus, rheumatoid arthritis, type 1 diabetes, multiple sclerosis, myasthenia gravis, Graves disease, pernicious anemia, scleroderma, psoriasis, inflammatory bowel diseases, Hashimoto's disease, Addison's disease, cutaneous autoimmune disease, systemic sclerosis and Sjögren's syndrome.

In these embodiments, the agents can be administered to the subject or the pDC cells can be treated, contacted or incubated with one of more agents per the methods of the invention ex vivo, and transplanted into the subject.

Systemic Sclerosis (SSc) is a multisystem, fibrosing disorder in which vasculopathy, autoimmunity, and inflammation lead to diverse life-altering and life-threatening clinical manifestations (Varga and Abraham 2007). SSc has the highest degree of morbidity and mortality of the rheumatic diseases with a ten-year mortality rate of 23% to 45% (Mayes et al. 2003). The female predominance is about 4:1, and the usual age of onset is 35 to 55 years of age. The pathophysiology of SSc is not completely understood, but substantial evidence shows interplay between immunologic derangement, endothelial dysfunction, and pro-fibrotic mechanisms.

As previously shown, pDCs infiltrate the skin of SSc patients and are chronically activated, leading to increased secretion of IFN-α and CXCL4 which are both hallmarks of the disease. pDC is an essential cell type involved in the pathogenesis of SSc and attenuating pDC function could be a novel approach to treat SSc patients.

Yet a further embodiment of the current disclosure is a method of treating and/or preventing systemic sclerosis in a subject in need thereof by administering a therapeutically effective amount of Neu5ac to the subject, wherein the administration of the agents decreases or inhibits the activation of pDCs to produce a lesser amount or level of IFN-α.

In these embodiments, the agents can be administered to the subject, preferably to the skin tissue directly or the pDC cells can be treated, contacted or incubated with one of more agents per the methods of the invention ex vivo, and transplanted into the subject, preferably to the skin tissue.

In these embodiments, the phrase “in need thereof” indicates a subject has an autoimmune disease, is suspected of having an autoimmune, or has risk factors for an autoimmune disease.

Additionally, PRP is used for a variety of indications including enhancing the healing process especially with regard to the healing of muscles, tendons and ligaments. PRP injections are also used to treat orthopedic and musculoskeletal injuries and conditions such as soft tissue injuries, tendonitis and osteoarthritis. As shown herein, N-Acetylneuraminic acid is found in the active fraction of the PCM and has the same anti-inflammatory effects of PCM/PRP. Thus, further embodiments of the current disclosure would include the use of N-Acetylneuraminic acid for any use currently PRP is currently indicated.

One embodiment is a method of treating musculoskeletal injuries and conditions in a subject in need thereof comprising administering a therapeutically effective amount of N-Acetylneuraminic acid.

Injuries would include but are not limited to soft tissue injuries including injuries to muscles, tendons and ligaments. Conditions would include but are not limited to osteoarthritis and tendonitis.

Also as shown herein, a particular active fraction of PCM and a salt in the active fraction of the PCM also have the same anti-inflammatory effects of PCM/PRP. Thus, further embodiments of the current disclosure would include the use of these for any use currently PRP is currently indicated.

One embodiment is a method of treating musculoskeletal injuries and conditions in a subject in need thereof comprising administering a therapeutically effective amount of the active fraction of PCM or a salt in the active fraction of the PCM.

Injuries would include but are not limited to soft tissue injuries including injuries to muscles, tendons and ligaments. Conditions would include but are not limited to osteoarthritis and tendonitis.

In any of these embodiments, the N-Acetylneuraminic acid, or the active fraction of the PCM can be administered with other therapeutic agents. In these embodiments, the N-Acetylneuraminic acid or the active fraction of the PCM and the therapeutic agents can be administered together or separately. In further embodiments, the pDC cells can be treated, contacted or incubated with one of more agents per the methods of the invention ex vivo, and transplanted into the subject.

### Pharmaceutical Compositions, Administration and Dosing

While it is possible that the N-Acetylneuraminic acid, or a pharmaceutically acceptable salt, derivative, or metabolite thereof, as well as salts, solvates and physiological functional derivatives thereof, may be administered as the raw chemical, it is possible to present the active ingredient as a pharmaceutical composition. It is also contemplated that the active fraction obtained from the PCM or a salt in the active fraction can be the active ingredient in a pharmaceutical composition. Accordingly, the disclosure further provides a pharmaceutical composition, which comprises the disclosed agents or compounds and/or salts, solvates and physiological functional derivatives thereof, and one or more pharmaceutically acceptable carriers, diluents, or excipients. The carrier(s), diluent(s) or excipient(s) must be acceptable in the sense of being compatible with the other ingredients of the formulation and not deleterious to the recipient thereof. In accordance with another aspect of the disclosure there is also provided a process for the preparation of a pharmaceutical composition including admixing the present compound, or salts, solvates and physiological functional derivatives thereof, with one or more pharmaceutically acceptable carriers, diluents or excipients.

The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound or compounds, and pharmaceutically acceptable excipients.

Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopoeia or other generally recognized pharmacopeias for use in animals, and more particularly in humans.

“Carrier” refers to a diluent, adjuvant, excipient, or vehicle with which the therapeutic is administered. Such pharmaceutical carriers can be sterile liquids, such as saline solutions in water and oils, including those of petroleum, animal, vegetable, or synthetic origin, such as peanut oil, soybean oil, mineral oil, sesame oil, and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents.

Preferred methods of administration include oral; mucosal, such as nasal, sublingual, vaginal, buccal, or rectal; parenteral, such as subcutaneous, intravenous, bolus injection, intramuscular, or intraarterial; or transdermal administration to a subject.

A further preferred form of administration is parenteral including injection and intravenous administration.

Pharmaceutical compositions adapted for parenteral administration, including intravenous administration, include aqueous and non-aqueous sterile injectable solutions or suspensions, which may contain anti-oxidants, buffers, bacteriostats, and solutes that render the compositions substantially isotonic with the blood of the subject. Other components which may be present in such compositions include water, alcohols, polyols, glycerine, and vegetable oils. Compositions adapted for parental administration may be presented in unit-dose or multi-dose containers, such as sealed ampules and vials, and may be stored in a freeze-dried (lyophilized) condition requiring only the addition of a sterile carrier, immediately prior to use. Extemporaneous injection solutions and suspensions may be prepared from sterile powders, granules, and tablets. Suitable vehicles that can be used to provide parenteral dosage forms of the invention are well known to those skilled in the art. Examples include: water for Injection USP; aqueous vehicles such as Sodium Chloride Injection, Ringer's Injection, Dextrose Injection, Dextrose and Sodium Chloride Injection, and Lactated Ringer's Injection; water-miscible vehicles such as ethyl alcohol, polyethylene glycol, and polypropylene glycol; and non-aqueous vehicles such as corn oil, cottonseed oil, peanut oil, sesame oil, ethyl oleate, isopropyl myristate, and benzyl benzoate.

In a further embodiment, the present disclosure provides a pharmaceutical composition adapted for administration by the oral route.

Pharmaceutical compositions of the present disclosure which are adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free-flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

It should be understood that, in addition to the ingredients particularly mentioned above, the compositions may include other agents conventional in the art having regard to the type of formulation in question, for example those suitable for oral administration may include flavoring agents.

Further methods of administration include mucosal, such as nasal, sublingual, vaginal, buccal, or rectal; or transdermal administration to a subject.

Pharmaceutical compositions adapted for nasal and pulmonary administration may comprise solid carriers such as powders, which can be administered by rapid inhalation through the nose. Compositions for nasal administration may comprise liquid carriers, such as sprays or drops. Alternatively, inhalation directly through into the lungs may be accomplished by inhalation deeply or installation through a mouthpiece. These compositions may comprise aqueous or oil solutions of the active ingredient. Compositions for inhalation may be supplied in specially adapted devices including, but not limited to, pressurized aerosols, nebulizers or insufflators, which can be constructed so as to provide predetermined dosages of the active ingredient.

Selection of a therapeutically effective dose will be determined by the skilled artisan considering several factors, which will be known to one of ordinary skill in the art. Such factors include the particular form of the N-Acetylneuraminic acid and other agents and their pharmacokinetic parameters such as bioavailability, metabolism, and half-life, which will have been established during the usual development procedures typically employed in obtaining regulatory approval for a pharmaceutical compound. Further factors in considering the dose include the condition or disease to be treated or the benefit to be achieved in a normal individual, the body mass of the patient, the route of administration, whether the administration is acute or chronic, concomitant medications, and other factors well known to affect the efficacy of administered pharmaceutical agents. Thus, the precise dose should be decided according to the judgment of the person of skill in the art, and each patient's circumstances, and according to standard clinical techniques.

In certain embodiments, the effective or therapeutically effective amount or dose may be adjusted depending on conditions of the disease/disorder to be treated or prophetically treated, the age, body weight, general health conditions, sex, and diet of the subject, dose intervals, administration routes, excretion rate, and combinations of drugs.

An initial dose may be larger, followed by one or more smaller maintenance doses. Other ranges are possible, depending on the subject's response to the treatment. An initial dose may be the same as, or lower or higher than subsequently administered doses.

The composition may be administered daily, weekly, biweekly, several times daily, semi-weekly, every other day, bi-weekly, quarterly, several times per week, semi-weekly, monthly, or more. The duration and frequency of treatment may depend upon the subject's response to treatment.

In certain embodiments, when there are more than one doses of the present composition administered to a subject, the second dose is lower than the first dose. In certain embodiments, the second dose is an amount that is at most one-half, one-quarter, or one-tenth the amount of the first dose.

The number and frequency of doses may be determined based on the subject's response to administration of the composition, e.g., if one or more of the patient's symptoms improve and/or if the subject tolerates administration of the composition without adverse reaction.

Treatment using the present method can continue as long as needed.

The co-administration of the agents can be by any administration described herein. Moreover, it can be in one composition, or in more than one composition. The administration of the agents can be simultaneous, concurrently or sequentially.

Alternatively, a further embodiment provides methods of ex vivo cell therapy, wherein a population of cells is obtained, contacted, incubated or treated with one of the agents disclosed herein, and then administered back to the subject in need thereof.

In some embodiments, the cells (pDCs) are obtained from a subject, such as a mammalian subject. In some embodiments, the mammalian subject is a non-human primate, a rodent (e.g., mouse or rat), a bovine, a porcine, an equine, or a domestic animal. In some embodiments, the cells are obtained from a healthy donor. In some embodiments, the cells are obtained from the subject to whom the treated cells will be subsequently administered. Cells that are administered to the same subject from which the cells were obtained are referred to as autologous cells, whereas cells that are obtained from a subject who is not the subject to whom the cells will be administered are referred to as allogeneic cells.

Cells may be obtained from any suitable source using convention means known in the art. In some embodiments, cells are obtained from a sample from a subject, such as skin sample or a blood sample.

pDCs can be mobilized into the circulating blood by administering a mobilizing agent in order to harvest the cells from the peripheral blood. The number of the cells collected following mobilization using a mobilizing agent is typically greater than the number of cells obtained without use of a mobilizing agent.

In some embodiments, a sample is obtained from a subject and is then enriched for a desired cell type. pDCs can be obtained from PBMCs isolated from blood. Cells can also be isolated from other cells, for example by isolation and/or activation with an antibody binding to an epitope on the cell surface of the desired cell type. Another method that can be used includes negative selection using antibodies to cell surface markers to selectively enrich for a specific cell type without activating the cell by receptor engagement.

Populations of the cells can be expanded prior to or after treatment, contact or incubation with the desired agent or agents. The cells may be cultured under conditions that comprise an expansion medium. The cell may be expanded for about 1, 2, 3, 4, 5, 6, 7, 8, 9, 10, 11, 12, 13, 14, 15, 16, 17, 18, 19, 20, 21, 22, 23, 24, 25 days or any range necessary.

The cells may be autologous to the subject, i.e., the cells are obtained from the subject in need of the treatment, treated, contact and/or incubated with the agents described herein, and then administered to the same subject. Administration of autologous cells to a subject may result in reduced rejection of the host cells as compared to administration of non-autologous cells. Alternatively, the host cells are allogeneic cells, i.e., the cells are obtained from a first subject, treated, contact and/or incubated with the agents described herein and administered to a second subject that is different from the first subject but of the same species. For example, allogeneic immune cells may be derived from a human donor and administered to a human recipient who is different from the donor.

A typical amount of cells, i.e., administered to a mammal (e.g., a human) can be, for example, in the range of one million to 100 billion cells; however, amounts below or above this exemplary range are also within the scope of the present disclosure. For example, the daily dose of cells can be about 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), preferably about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), more preferably about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells, or a range defined by any two of the foregoing values).

Also within the scope of the present disclosure are multiple administrations (e.g., doses) of the agents and/or populations of cells. In some embodiments, the agents and/or populations of cells are administered to the subject once. In some embodiments, agents and/or populations of cells are administered to the subject more than once (e.g., at least 2, 3, 4, 5, or more times). In some embodiments, the agents and/or populations of cells are administered to the subject at a regular interval, e.g., every six months.

The present disclosure further provides for pharmaceutical compositions comprising the cells treated, incubated or contacted with agents using the methods disclosed herein for the treatment of disease. These cells include plasmacytoid dendritic cells treated, incubated or contacted with agents using the methods disclosed herein.

### Kits

Also within the scope of the present disclosure are kits for practicing the methods disclosed herein.

In some embodiments, the kit can comprise instructions for use in any of the methods described herein. The included instructions can comprise a description of administration of the agents to a subject to achieve the intended activity in a subject. The kit may further comprise a description of selecting a subject suitable for treatment based on identifying whether the subject is in need of the treatment.

The instructions relating to the use of the N-Acetylneuraminic acid or other agents or compositions described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### Methods to Obtain the Active Fraction of PRP/PCM

As discussed herein, PRP/PCM is used for many indications with varying results. Shown herein are methods to obtain an active fraction of PRP/PCM. This active fraction has anti-inflammatory properties and the ability to decrease an immune response. Given the mixed results using PRP/PCM, it is extremely useful to obtain an active fraction with a concentration of beneficial molecules and factors.

The first disclosed method comprises the steps of:
A. fractionating the PCM to obtain a water phase:
B. extracting the water phase using DMF under acidic condition, to obtain a solid acidic fraction; and
C. resolubilizing the solid acidic fraction in acetonitrile/methanol/H₂O (40/40/20) and collecting the soluble fraction to obtain the active fraction.

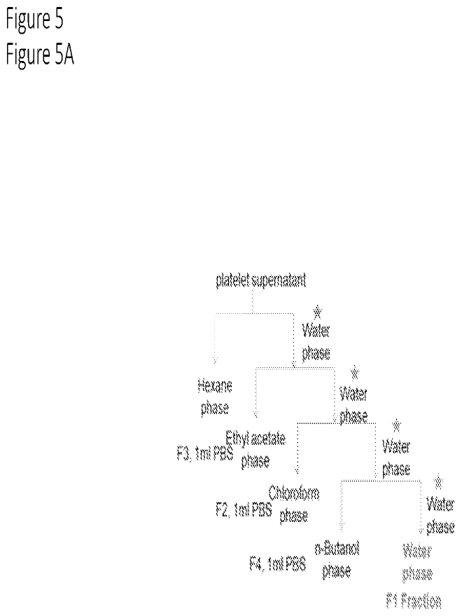

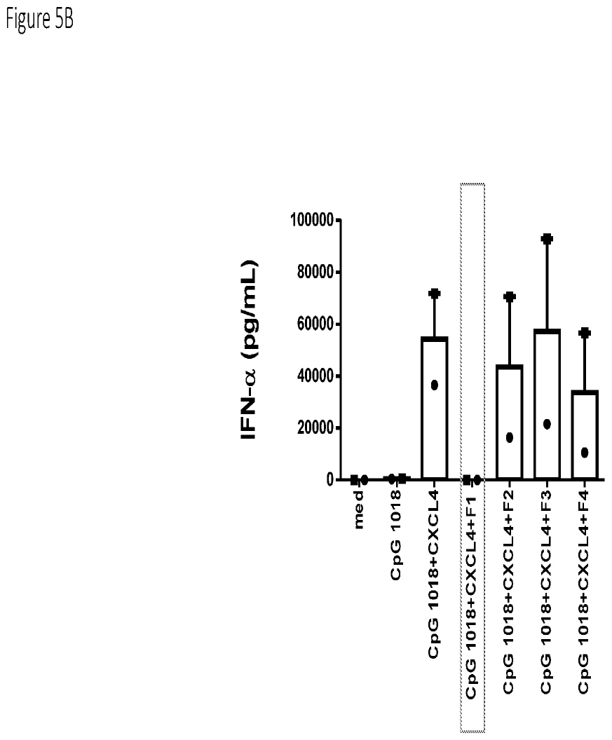

### See FIGS. 5, 6, and 8.

Step A of this method includes several steps of fractionating the PCM to obtain a water phase. This can be accomplished by any method known in the art including but not limited to reverse phase HPLC and anion exchanger column.

Step B of the method requires the extraction of the water phase to obtain a solid acidic fraction. Again this can be done by any method known in the art.

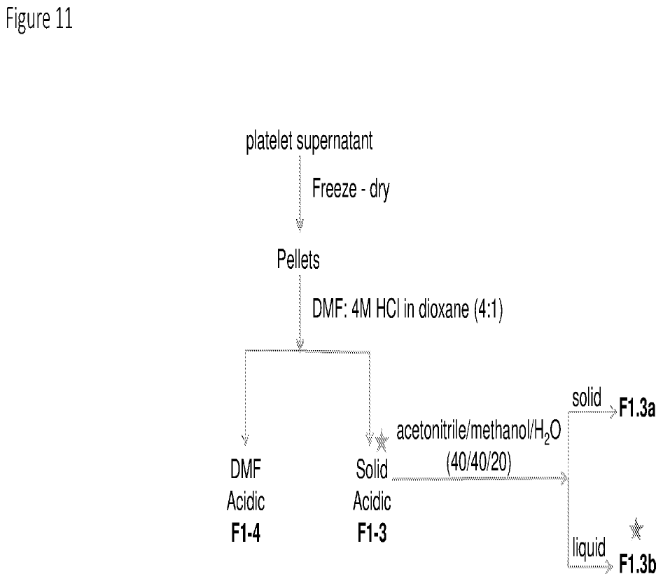

Steps A and B of the foregoing method, while effective, are labor intensive and time consuming as several steps of fractionating and collecting the phases on various columns, as well as concentrating, re-suspending and lyophilizing, must be performed. Because of this, a more streamlined and efficient method was developed to obtain the active fraction. This method comprises the steps:
A. adding a mixture of DMF and 4M HCl in Dioxane at 4:1 by volume to freeze-dried platelet supernatant pellets and removing a soluble fraction to obtain a solid acidic fraction; and
B. resolubilizing the solid acidic fraction in acetonitrile/methanol/H₂O (40/40/20) and collecting the soluble fraction to obtain the active fraction.
See FIG. 11.

It was determined that the water-soluble active component could be found in the freeze-dried pellet, thus, freeze-drying the PCM or using freeze-dried PCM would allow an easy approach to obtaining the active water phase. It was also found that DMF could remove all the inactive ingredients that were removed by less polar solvents.

Thus, this streamlined method requires only adding a mixture of DMF and HCl to freeze-dried platelet supernatant pellets, and then resolubilizing the solid in acetonitrile/methanol/H₂O and collecting the soluble active fraction. This freeze-dried platelet supernatant pellets can be obtained by freezing the platelet supernatant and then freeze-drying it to form pellets, or the pellets can be obtained through other sources, such as commercial and research.

This method requires less steps meaning less active material is lost and lower time and costs. It produces the same active fraction of the PCM as the more complicated labor-intensive method.

### EXAMPLES

The present invention may be better understood by reference to the following non-limiting examples, which are presented in order to more fully illustrate the preferred embodiments of the invention. They should in no way be construed to limit the broad scope of the invention.

### Example 1-PCM Exerts an Anti-Inflammatory Effect on pDCs Via a Molecule Less than 5 kDa

### Materials and Methods

### Preparation of Initial PCM Fractions

Platelets were either isolated from fresh blood or obtained from the New York Blood Center. Isolated platelets were resuspended in PBS 1× in the presence of Mg2+& Ca2+ at a concentration of 100 million cells/100 μl PBS and cultured for 24 hours.

After 24 hours, the platelets were centrifuged and the supernatant or platelet-conditioned media (PCM) were collected. To remove lipids, the PCM was loaded on captiva EMR-Lipid cartridges (Agilent technologies) and eluted by applying positive pressure with a 3 ml syringe plunger. Then, nucleic acids were removed by treating the PCM with RNase (5 μg/ml) and DNase (2000 U) for 15 minutes at RT. The PCM was then treated with Proteinase K (0.5 mg/ml) for 10 minutes at 55° C. and RNase, DNase and proteinase K were heat-inactivated for 30 minutes at 95° C. Finally, the PCM was separated in less than (<) and greater than (>) 5 kD by centrifugation (30 minutes at 15000 g in 5 kD concentrators (Corning Spin-X UF concentrators, 5K)).

Purification of Cells from Healthy Donors

PBMCs were prepared using Ficoll-Paque density gradient from fresh blood under internal Institutional Review Board-approved protocols. pDCs were isolated as previously described (Guiducci et al. 2008) using either using negative depletion (Miltenyi Biotech) or following cell sorting using a BD IFNLUX cell sorter. All BDCA4+ cells also express BDCA2 (results not shown). As a result, pure pDCs were obtained and no markers for neutrophils (CD66) or myeloid cells were found by PCR.

### TLR9 Activation

For the TLR9 activation in pDCs, the cells were incubated in complete media alone, or CpG-B 1018 (0.25 or 1 μM), or CpG-C 274 at similar concentrations. In some assays, cells were also incubated with 10 μg/ml CXCL4 (R&D systems).

### Real-Time Quantitative PCR Analysis

PCR reactions were performed as described previously with 10 ng of cDNA (Barrat et al. 2005). In brief, RNA was extracted from cells using the Qiagen RNeasy Mini Kit. Quantity of RNA was measured by Nanodrop and high-capacity cDNA Reverse Transcription kit was used to generate 20-50 ng cDNA. Gene expression levels were calculated based on relative threshold cycle (Ct) values. This was done using the formula Relative Ct=100×1.8 (HSK-GENE), where HSK is the mean CT of duplicate housekeeping gene runs (ubiquitin or GADPH), GENE is the mean CT of duplicate runs of the gene of interest, and 100 is arbitrarily chosen as a factor to bring all values above 0.

### Measurement of Inflammatory Proteins

Supernatant from B cells and pDCs 24 hour culture was used for quantification of secreted IFNα with the use of an enzyme-linked immunosorbent assay (ELISA) (Mabtech) performed according to the manufacturer's protocol.

### Statistical Analysis

Data were analyzed using a Mann-Whitney U-test (t test using non parametric criteria for independent samples). Comparisons between PBMC and pDCs-depleted PBMC were tested using a parametric paired t-test. All analyses were performed using Prism software (GraphPad Software). Differences were considered significant at a P level less than 0.05 with *p<0.05; **p<0.01; ***p<0.001.

### Results

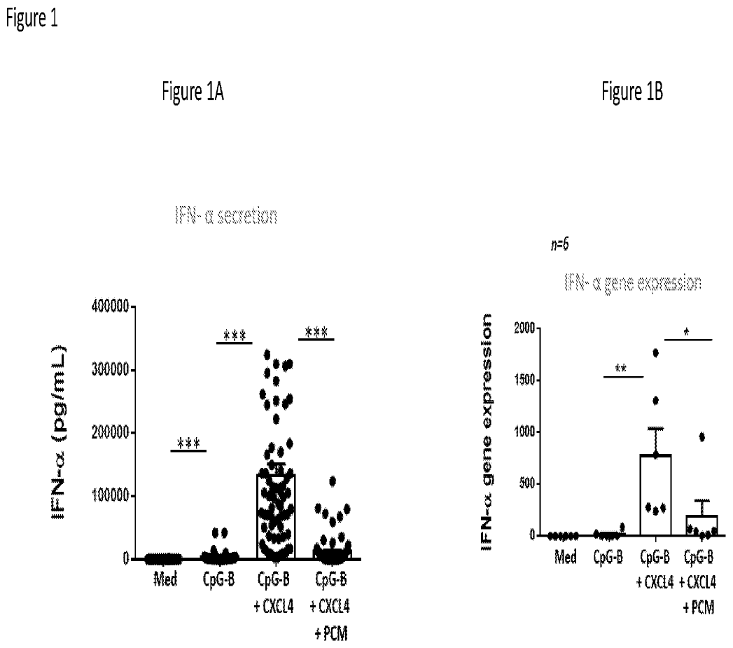

As shown in FIG. 1, PCM inhibits the expression and secretion of interferon-α in CXCL4+TLR9-activated pDCs. Specifically, the secretion and expression of IFN-α was inhibited in pDCs which were stimulated with CXCL4 and CpG-B, a TLR9 ligand (FIGS. 1A and 1B).

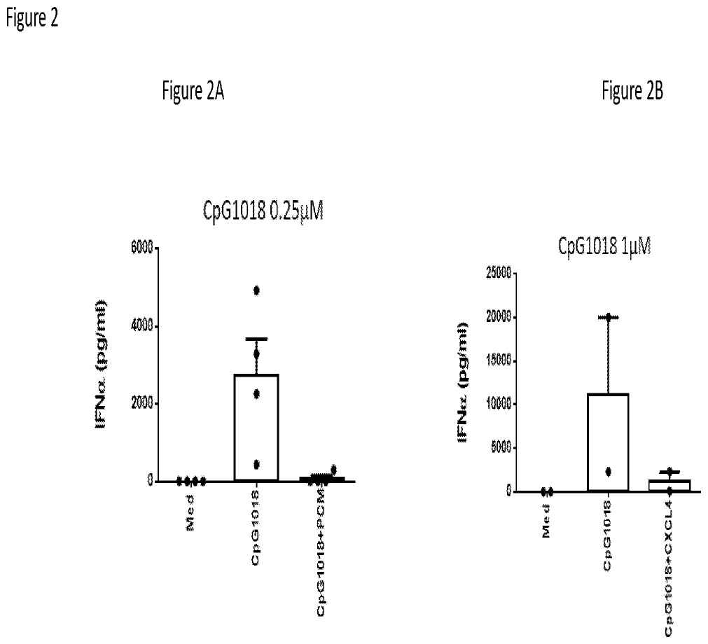

PCM further inhibits the expression and secretion of interferon-α in TLR9-activated pDCs. Specifically, the secretion and expression of IFN-α was inhibited in pDCs which were stimulated CpG1018 (FIGS. 2A and 2B).

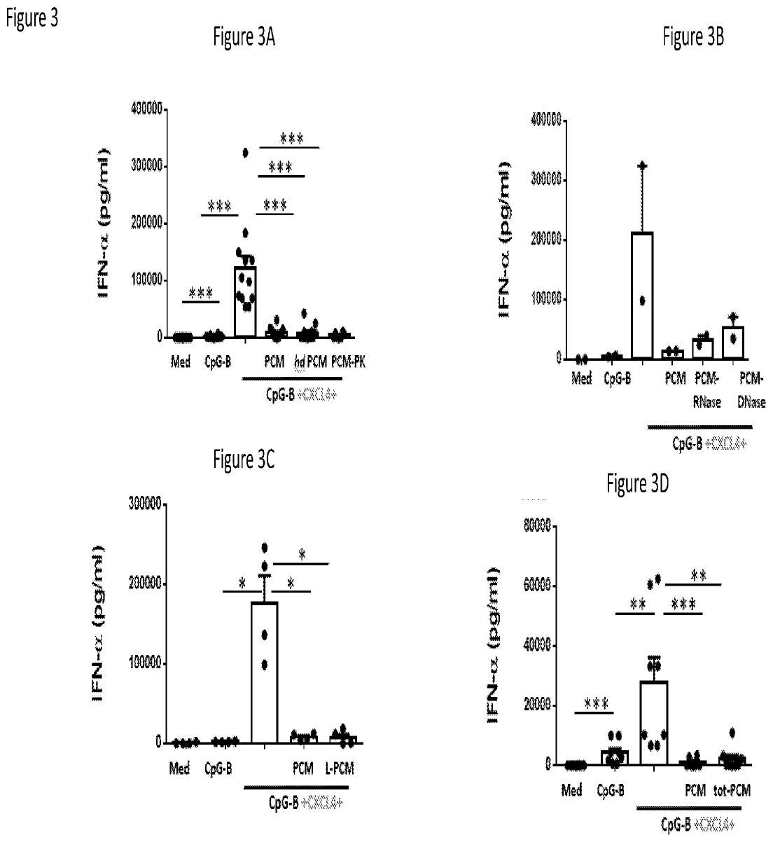

The active molecule in PCM that inhibits the CXCL4 effect in the TLR9-induced pDCs is not a protein as proteinase K digestion and heat denaturation had no effect on the inhibitory activity of the PCM (FIGS. 3A and 3D). The active molecule is also not a nucleic acid as RNase A and DNase treatment of the PCM had no effect on the inhibitory activity of the PCM (FIGS. 3B and 3D). The active molecule is also not a lipid as the removal of lipids from the PCM via agilent column had no effect on the inhibitory activity of the PCM (FIGS. 3C and 3D).

In order to further characterize the molecule, a fractionation was performed using size exclusion filters on the PCM which separated the PCM into two different fractions one less than 5 kDa and one more than 5 kDA.

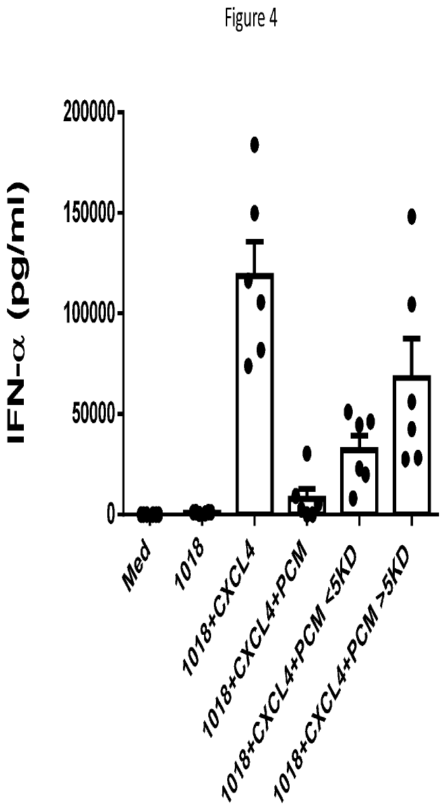

IFN-α secretion was only inhibited in the fractions that contained molecules under 5 kDa (FIG. 4).

Thus, it was concluded that the molecule which exerts the anti-inflammatory effect was under 5 kDa.

### Example 2—Fractionation of PCM to Obtain the Active Water Phase

### Materials and Methods

Random platelets were obtained from eight donors from the New York Blood Center and the combined less than 30 kD PCM (42 ml) was collected and fractionated as follows (see FIG. 5A):

first fraction was run on a preparative reversed phase (C18 column) HPLC using the following parameters:
Column: prep C18 5 μM, 19*150 mm column
Mobile phase: water and acetonitrile without formic acid
Methods: 0_0.5 min 5% acetonitrile
   0.5_10.0 min 5%_95% acetonitrile gradient
   10.0_13.0 min 95% acetonitrile
Injection: 900 uL
Flow rate: 13 mL/min
Collection: collect in 20 tubes according to time event, combine each 2 tubes, and get 10 fractions
Concentration: lyophilization at −80° C. and 0.09 mBar for 3 days.

The active metabolite was eluted with 5%-10% acetonitrile suggesting that the active metabolite is highly polar and cannot bind to the C18 column.

The second fraction was run on a strong anion exchanger (SAX) column using the following parameters and procedure:
Column: strong anion exchanger (SAX), 3 mL, 500 mg (Agilent technologies, Part No: 12102044)

Sample pretreatment: 100 uL sample+200 uL dd H₂O+10 uL 5% NH₃ to adjust the pH to 7-11
Procedure: 1. Column conditioning: 4 mL methanol, then 4 mL H₂O
   2. Sample loading: the prepared sample is passed through the column by pressure
   3. Fraction 1: elution with 4 mL 5% NH₃ in water
   4. Fraction 2: elution with 4 mL methanol_H₂O (¼, v/v)
   5. Fraction 3: elution with 4 mL methanol_acetone (1/1, v/v)+5% AcOH
   6. Fraction 3: elution with 4 mL methanol_acetone (1/1, v/v)+5% AcOH
Concentration: 1. Concentrate each fraction on rotary evaporator for 30 min at 30° C. and 4 mBar
   2. resuspend the residue in 4 mL dd water
   3. lyophilization at −80° C. and 0.09 mBar for 2 days

All fractions were re-suspended in 1 ml of PBS and tested for activity by measuring inhibition of IFN-α in TLR9 activated pDCs as described in Example 1.

### Results

As shown in FIG. 5, the active metabolite was always found in the water phase. FIG. 5B shows that the water phase designated fraction F1 inhibited activated pDCs and IFN-α production.

### Example 3—Fractionation of the Active Water Phase

### Materials and Methods

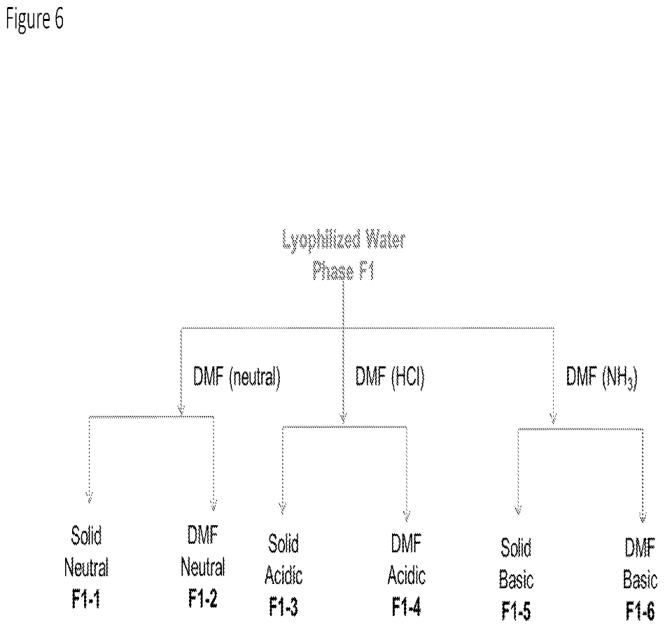

The active water phase fraction was then split into four parts and lyophilized and further extracted as follows (see FIG. 6):
1. The first part of the lyophilized sample was extracted with DMF under neutral conditions to obtain two fractions: solid neutral (F1-1) and DMF neutral (F1-2).
2. The second part of the lyophilized sample was extracted with DMF: 4M HCl in dioxane (4:1) to obtain two fractions: solid phase acidic (F1-3) and DMF phase acidic (F1-4).
3. The third part of the lyophilized sample was extracted with DMF: 4M NH₃ in DMF (4:1) to obtain two fractions: solid basic (F1-5) and DMF basic (F1-6).
F1-1, 1-2, 1-3 and 1-4 were done together, and F1-5 and 1-6 were done later

All sub-fractions were lyophilized and then resuspended in PBS and tested for activity by measuring inhibition of IFN-α in TLR9 activated pDCs as described in Example 1.

### Results

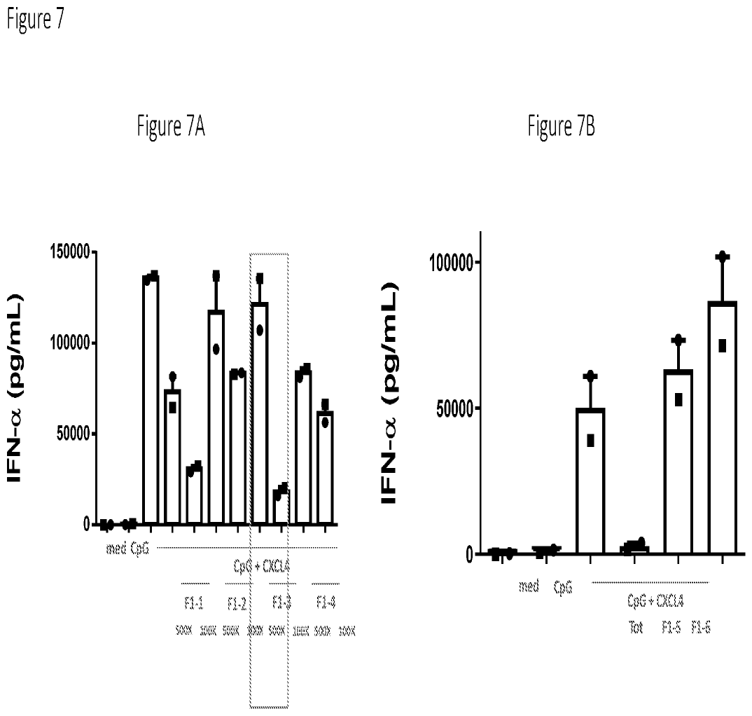

As shown in FIGS. 7A and 7B, both the solid neutral fraction (F1-1) and the solid acidic fraction (F1-3) were active, but the solid acidic fraction was the most active.

### Example 4—Further Fractionating and Testing of the Solid Acidic Fraction F1-3

### Materials and Methods

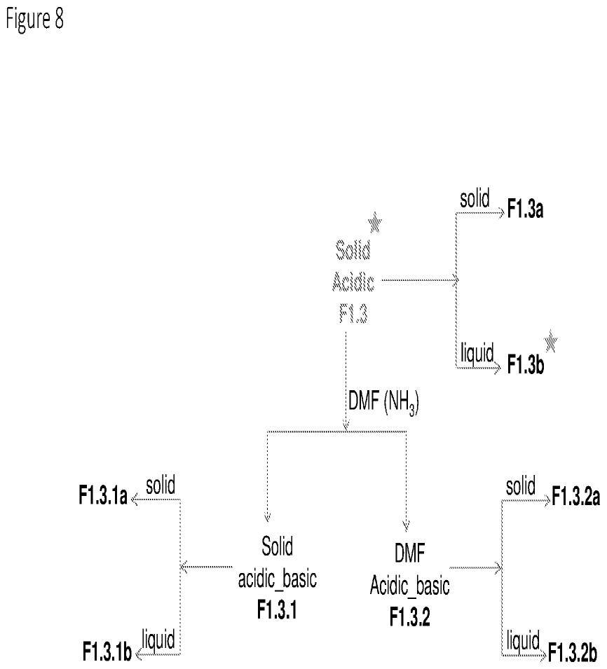

The solid acidic fraction F1-3 from Example 3 was further fractionated. The fraction was lyophilized and the first part of the lyophilized sample was extracted with DMF under neutral conditions to obtain two fractions: solid (F1.3a) and liquid (F1.3b). The second part of the lyophilized sample was extracted with DMF: 4M NH₃ in DMF (4:1) to obtain two fractions: solid acidic basic (F1.3.1) and DMF acidic basic (F1.3.2). Fractions F1.3, 1.3.1 and 1.3.2 were lyophilized and desalted using solvent acetonitrile/methanol/H₂O (40/40/20). See FIG. 8.

Fractions soluble in methanol/acetonitrile were fractions a, and salt were in b

All sub-fractions were lyophilized and then resuspended in PBS and tested for activity by measuring inhibition of IFN-α in TLR9 activated pDCs as described in Example 1. Sub-fractions 1.3a and 1.3b were diluted from 100 to 10 for 1.3a and 10,000 to 100 for 1.3b and tested for activity.

### Results

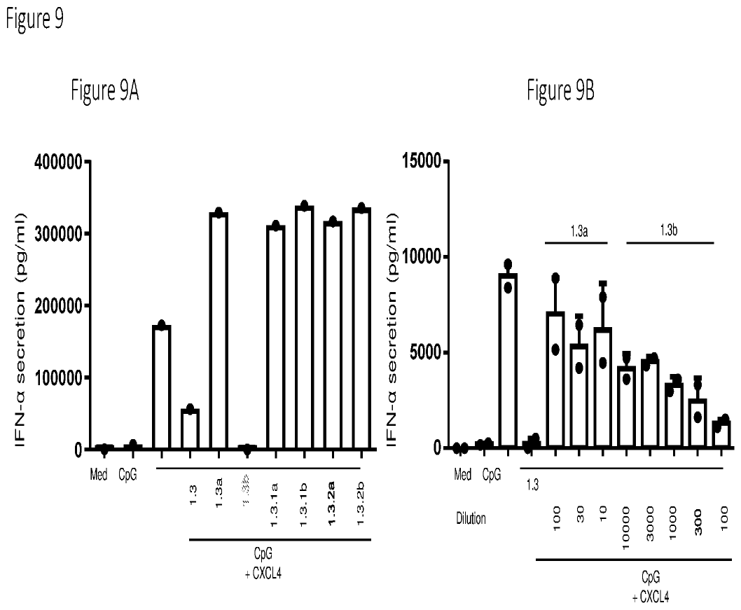

As shown in FIGS. 9A and 9B, the active fraction in F1.3 is most likely a salt as fraction 1.3b had the inhibitory activity.

### Example 5—Identification of N-Acetylneuraminic Acid as an Active Molecule

### Materials and Methods

Metabolites/molecules present in the active fraction F1.3b were analyzed by liquid chromatography-mass spectrometry (LC-MS) and compared to those in the non-active fractions F1.3.b and F1.3.2b using the assay described in Example 1.

Among the small molecules tested were: N-Acetylneuraminic acid; 2MC: 2-methylcitric; Ch3Ala: α-Methyl-phenylalanine; NAAG: aspartyl glutamate; acid αKG: oxoglutaric acid (alpha-ketoglutaric acid); Phosphorylcholine; 3-Phosphoglyceric acid; Galactonic acid; gluconic acid; taurine; and N-Acetyl-L-aspartic acid.

### Result

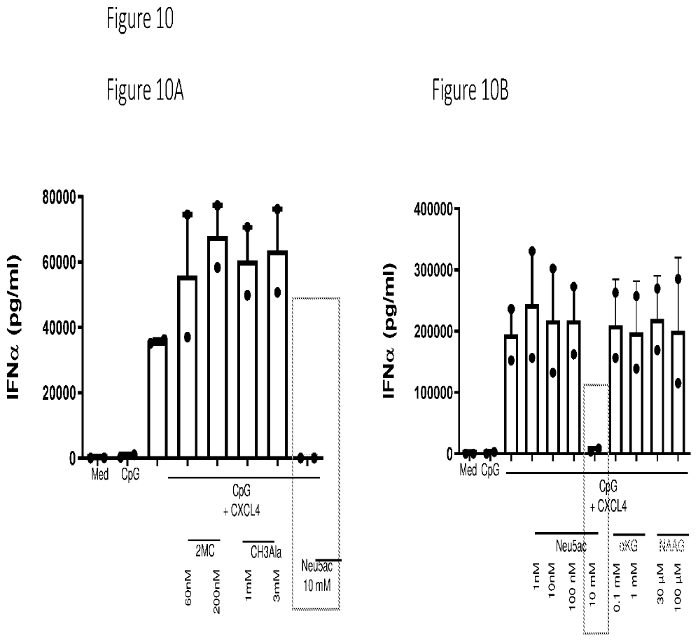

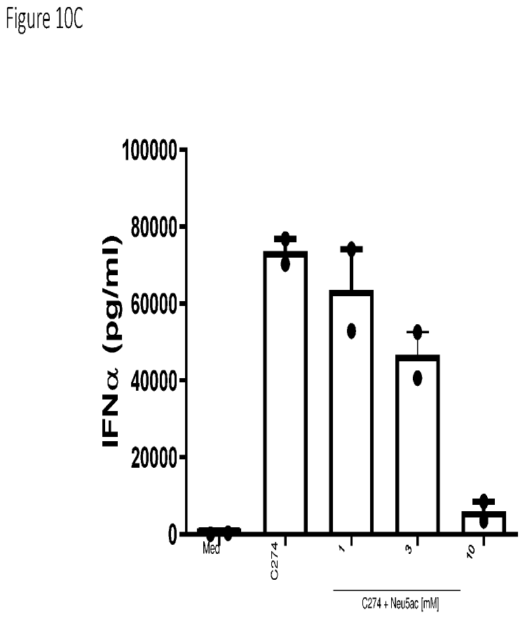

As shown in FIG. 10, only N-Acetylneuraminic acid had a significant inhibitory effect on the TLR9-activated pDCs (with or without CXCL4 present), showing that N-Acetylneuraminic acid has this inhibitory/anti-inflammatory activity of PCM. (Results not shown for Phosphorylcholine; 3-Phosphoglyceric acid; Galactonic acid; gluconic acid; taurine; and N-Acetyl-L-aspartic acid).

### Example 6-Alternative Method to Obtain the Active Fraction of PCM/PRP

An alternative method to obtain active fraction F1.3b was developed and is as follows:
1) The 10 mL platelet supernatant was frozen to −80° C. and freeze-dried on Labconco lyophilizer at −80° C. for 24h providing pellets.
2) 2 mL mixture of DMF and 4M HCl in dioxane (4/1, v/v) was added to the pellets. After vortex and centrifugation, the supernatant was discarded. The pellet was washed with 2 mL×2 mixture solvent of DMF and 4M HCl in dioxane (4/1, v/v). The collected pellet was dissolved in 2 mL ddH₂O and freeze-dried on Labconco lyophilizer at −80° C. for 24h to remove residual DMF and dioxane.
3) The resulting pellet F1-3 was re-solubilized in 2 mL acetonitrile/methanol/H₂O (40/40/20, v/v/v). After vortex and centrifugation, the supernatant was collected. The pellet was washed with 2 mL×2 acetonitrile/methanol/H₂O (40/40/20, v/v/v). The supernatant was combined and the pellet discarded. Most of acetonitrile and methanol in the supernatant was removed using rotary evaporator. The concentrated supernatant was freeze-dried on Labconco lyophilizer at −80° C. for 24h providing the fraction F1.3b.

### See FIG. 11.

### REFERENCES

Barrat and Su, A Pathogenic Role of Plasmacytoid Dendritic Cells in Autoimmunity and Chronic Viral Infection. J Exp Med 216, 1974-85 (2019).
Guiducci et al., RNA recognition by human TLR8 can lead to autoimmune inflammation. J Exp Med 210, 2903-2919 (2013).
Wenzel and Tuting, An IFN-Associated Cytotoxic Cellular Immune Response against Viral, Self-, or Tumor Antigens Is a Common Pathogenetic Feature in “Interface Dermatitis”. J Invest Dermatol 128, 2392-2402 (2008).

1-9. (canceled)

10. A method of obtaining an active fraction of platelet-conditioned media, comprising the steps of:
A. adding a mixture of DMF and 4M HCl in Dioxane at 4:1 by volume to freeze-dried platelet supernatant pellets and removing a soluble fraction to obtain a solid acidic fraction; and
B. resolubilizing the solid acidic fraction in acetonitrile/methanol/H₂O (40/40/20) and collecting the soluble fraction to obtain the active liquid fraction.

11. A method of inhibiting or decreasing an immune response in a subject in need thereof comprising administering to the subject a therapeutically effective amount of the active fraction obtained in claim 10.

12. A method for inhibiting or decreasing inflammation in a subject in need thereof comprising administering a therapeutically effective amount of the active fraction obtained in claim 10.

13. A method for inhibiting or decreasing the production of interferon-α in plasmacytoid dendritic cells in a subject in need thereof comprising administering a therapeutically effective amount of the active fraction obtained in claim 10.

14. A method of preventing and/or treating a condition, disease or injury in a subject in need thereof comprising administering a therapeutically effective amount of the active fraction obtained in claim 10, wherein the active fraction inhibits or decreases the expression or production of interferon-α by plasmacytoid dendritic cells in the subject.

15. The method of claim 14, wherein the disease is an autoimmune disease.

16. The method of claim 15, wherein the autoimmune disease is selected from the group consisting of systemic lupus erythematosus, rheumatoid arthritis, type 1 diabetes, multiple sclerosis, myasthenia gravis, Graves disease, pernicious anemia, scleroderma, psoriasis, inflammatory bowel diseases, Hashimoto's disease, Addison's disease, cutaneous autoimmune disease, systemic sclerosis, and Sjögren's syndrome.

17. The method of claim 14, wherein the condition is musculoskeletal.

18. The method of claim 17, wherein the musculoskeletal condition is selected from the group consisting of osteoarthritis and tendonitis.

19. The method of claim 14, wherein the injury is selected from the group consisting of injuries to muscles, tendon and ligaments.
